(19) United States
(12) Patent Application Publication
(10) Pub. No.: US 2005/0037025 A1
(43) Pub. Date: Feb. 17, 2005

(54) Methods and compositions comprising kava and mate' or theobromine

(76) Inventors: Robert T. Gow, Naples, FL (US); John Pierce, Thousand Oaks, CA (US); George W. Sypert, Naples, FL (US)

Correspondence Address: TROUTMAN SANDERS LLP, BANK OF AMERICA PLAZA, SUITE 5200, 600 PEACHTREE STREET , NE, ATLANTA, GA, 30308-2216, US

(21) Appl. No.: 10/945,108

(22) Filed: Sep. 20, 2004

Related U.S. Application Data
(63) Continuation-in-part of application No. 10/263,579, filed on Oct. 3, 2002.
(63) Continuation-in-part of application No. 10/273,943, filed on Oct. 18, 2002.
(63) Continuation-in-part of application No. 10/408,900, filed on Apr. 8, 2003.
(63) Continuation-in-part of application No. 10/273,981, filed on Oct. 18, 2002.
(60) Provisional application No. 60/514,187, filed on Oct. 24, 2003.

(51) Int. Cl. A61K 35/78; A61K 31/366

(52) U.S. Cl. 424/195.18; 424/734; 514/460

(57) ABSTRACT

The present invention comprises compositions comprising combinations of compositions of extracts of kava mate' extract compositions or theobromine. The invention further comprises methods for treating conditions related to mental and physical fatigue, anxiety, muscle tension, nervous depression, headache, obesity, and mild pain as well a enhancement of cognition and mental focus comprising administering effective amounts of the compositions of the present invention.

### CROSS-REFERENCE TO RELATED APPLICATIONS

This application is a continuation-in-part of U.S. patent application Ser. No. 10/263,579, filed Oct. 3, 2002, which claims priority of U.S. Provisional Patent Application Nos. 60/326,928, filed Oct. 3, 2001, and 60/369,889, filed Apr. 3, 2002; and also is a continuation-in-part of U.S. patent application Ser. No. 10/273,943, filed Oct. 18, 2002, which is a continuation-in-part of U.S. patent application Ser. No. 10/263,579, filed Oct. 3, 2002, and is also a continuation-in-part of U.S. patent application Serial No. 10/408,900, filed Apr. 8, 2003, and U.S. patent application Ser. No. 10/273,981, filed Oct. 18, 2002, and claims priority to U.S. Provisional Patent Application Ser. No. 60/514,187, filed Oct. 24, 2003. The entire contents of these applications are hereby expressly incorporated by reference.

### FIELD OF THE INVENTION

The invention relates to methods and compositions comprising kava extract compositions in combination with mate' extract compositions, or with theobromine. Such compositions can comprise kavalactone compounds or alkaloid compounds in ratios that are not found in native plant materials. Such methods and composition provide beneficial health conditions while reducing the side effects associated with consumption of native plant materials.

### BACKGROUND OF THE INVENTION

Ilex paraguariensis, a perennial tree, is native to South America and is also known as mate' or yerba mate'. The genus Ilex is a member of the holly family, Aquifoliaceae, and is found worldwide in subtropical and tropical regions of both hemispheres. The most commercialized plant of South America, I. paraquariensis is used to prepare a tea-like beverage, better known as mate' beverage. The mate' beverage is made from the dried, toasted and milled leaves and stems of the plant genus and is widely consumed in Argentina, Paraguay, Uruguay, Southern Brazil and more recently in North America, Europe, and the Middle East.

The mate' beverage is consumed primarily as an infusion, either by the addition of boiling water to the dry plant material, or by repeated additions of near-boiling water to the dry plant material. This infusion allows for extraction of water soluble plant constituents. A large number of people regularly consume some amount of mate' beverage. In South America for example, approximately 30% of the population drink more than one liter per day of mate' beverage. Many people use the beverage as a treatment for various conditions such as mental and physical fatigue, headaches, weight loss, nervous depression, rheumatic pains, and improved cognition.

Consequently, one outcome of consumption of mate' beverage and conventional nutraceutical extractions, is the presence of caffeine-related disorders such as gastrointestinal problems, caffeine toxicity, jitteriness, generalized anxiety, and insomnia. The consumption of caffeine exaggerates stress and stress-related hormone release. Blood pressure is elevated and the risks for heart attack and stroke are increased when caffeine is routinely consumed. Because mate' beverage is such a part of the social structure and cultural habits of so many people and the fact that it is being increasingly used as a nutraceutical medicinal agent, reducing the amount of mate' consumed does not appear to be a viable method for reducing caffeine consumption. An additional problem is that the mate' that is consumed is made from raw mate' plant material, which has varying amounts of caffeine when consumed drink to drink or dose to dose. This variability can cause confusing symptoms in users, hence, making the diagnosis of physiological problems difficult. In addition, such variability can cause uneven results when mate' is used for treatment of various physiological conditions. A final issue regarding mate' is that clinical and epidemiologic studies have found a positive association between mate' consumption and cancer of the esophagus, oral cavity, pharynx, larynx, stomach and bladder.

Theobromine is best known for its effects in chocolate products. Theobromine has been synthesized and has been used as a drug to treat different medical conditions. For example, theobromine has been used as a diuretic making it particularly useful after a person has experienced cardiac failure. Cardiac failure often results in an excess accumulation of bodily fluids. Theobromine is also known for its ability to dilate blood vessels making it a commonly prescribed treatment for people suffering from high blood pressure. In addition, theobromine is known as a weak stimulant but does cause the jitteriness and hyper-anxiety associated with caffeine. As a stimulant, it has been noted to raise levels of serotonin making it an inexpensive anti-depressant. Theobromine also is an appetite suppressant and a useful adjunct for weight reduction. Theobromine remains in the body for a very long period of time. The half life after ingestion is approximately 6 hours. Another rather unique property of theobromine is its ability to relax bronchi in the lungs, which also has been used to treat asthmatic and pulmonary diseases. Theobromine has also been found to be relatively harmless in humans unless taken in excessive quantities.

Kava plant, a type of pepper plant also known as Piper methysticum, is generally found in Polynesia, Melanesia, and Micronesia. The kava plant contains high concentrations of active kavalactones (sometimes referred to as kavapyrones), including kavain, methysticin, yangonin, dihydro methysticin, demethoxyyangonin, and dihydrokavain. Kava has long been used as an herbal medicine for the treatment of, among others, mental and physical fatigue, tension, cognitive impairment, nervous depression, headaches, weight loss, and pain. The prized part of the kava plant is the root system because it contains the highest concentrations of the active kavalactones, although kavalactones are also found in other parts of the plant at lower concentrations.

To harvest the kavalactones from the kava plant, the root of the plant is conventionally processed to generate a consumable product. In conventional and historical methods, the root is dried and ground to a powder. This powder contains not only the kavalactones, but also plant oils, tannins, resins, and other substances. The powder can be mixed with water to form a beverage or can be packaged into capsules or other forms for oral delivery.

The effects of ingesting kava root vary from person to person. Common effects include a state of relaxation and a reduction in muscle tenseness, and it can also produce a mild state of euphoria. Kava root extracts have also been used to help sufferers of insomnia. The ratios of the various concentrations of kavalactones in the consumed kava product have impacts on the physical effects experienced by the user. There are a large number of different kava cultivars, each of which has differing ratios of the active kavalactones.

Health issues associated with consuming kava have recently arisen. There have been several reports suggesting a link between kava use and liver damage. One theory suggests that a toxic chemical (pipermethystine) may be present in the stem peelings and leaves of the kava plant, but not the root. As the demand for kava increased a few years ago, companies started buying the stem peelings and leaves along with the root, leading to ingestion of pipermethystine by kava users. Another theory is that two active kalalactones, methysticin and dihydromethysticin, which contain a methylenedioxyphenyl functional group, may form intermediary compounds that can inactivate multiple P450 enzymes. P450 enzymes can interfere with the metabolism of many pharmaceuticals which, under certain circumstances, could lead to liver toxicity.

One solution to these health problems is to limit the extraction of kavalactones to only the root, thus eliminating the presence of pipermethystine. Another solution is to develop new kava cultivars with reduced amounts of methysticin and dihydromethysticin and increased kavain content. Although such cultivars exist, they are very fragile, time consuming to maintain and not assured of success.

What is needed then are compositions of combinations of extracts of kava and extracts of mate', or extracts of kava and theobromine, that provide desired physiological effects and have lowered amounts of carcinogens, such as tannins. Such compositions would have medicinal value in improving mental focus and cognition, enhancing a sense of well being, reducing physical and mental fatigue and in treating obesity.

### SUMMARY OF INVENTION

The present invention comprises methods and compositions of kava extracts combined with mate' extracts, or with theobromine. Such compositions are provided for oral delivery systems in the form of tablets, capsules, lozenges, liquids, and emulsions to achieve a beneficial effect with a corresponding reduced incidence of dose related side effects. Kavalactones have been shown to have potentiating effects on other pharmacological compounds such as barbiturates and diazepams. The present invention is directed to providing the potentiating effect of kava extract compositions which enhances the desired effects of the mate' extract compositions or the theobromine compositions.

An aspect of the present invention comprises methods of selective extraction of compounds such as tannins from extraction compositions of mate' and kava to yield compositions having differing characteristics, such as a lower risk of cancer, than the native plant materials. Another aspect of the present invention comprises compositions that are combinations of compositions of kava extracts with mate' extracts or combinations of kava extract compositions and theobromine.

An aspect of the present invention comprises methods and compositions comprising kava and mate'. Methods of the invention comprise methods of extraction of compounds from plant source material of Piper methysticum, or kava, and mate', methods of making pharmaceutical or nutriceutical products comprising kava and mate', and methods of use of the extracted products, and pharmaceutical and nutriceutical products. Compositions of the present invention comprise extraction products of kava comprising extracted kavalactones that have altered kavalactone profiles that are not found in natural plant material, combined with compositions comprising compounds isolated from the plant material of mate' or compositions comprising theobromine. An embodiment of the compositions of the present invention comprises compositions comprising an extract of kava having reduced levels of methysticin and dihydromethysticin and increased levels of kavain and dihydrokavain, in combination with compositions of mate' that have reduced amounts of caffeine when compared to native mate' plant materials, or the kava compositions in combination with theobromine. Compositions of the present invention also comprise pharmaceutical and nutriceutical compositions such as a rapid-dissolving tablet containing a combination of extracts of kava and extracts of mate, or combinations of extracts of kava and theobromine.

An aspect of the present invention also comprises methods of selective extraction of compounds such as tannins from extracts of kava or mate' to yield compositions that have a lowered risk of cancer than the native plant materials. Another aspect of the present invention comprises compositions comprising extraction products of kava combined with extraction products of mate' having alkaloid compositions that are not found in native plant material, and that have reduced potential liver toxicity.

Another aspect of the present invention comprises methods for making compositions comprising kava extract compositions combined with mate' extract compositions that have a predetermined characteristic, such as altered kavalactone profiles, a lowered amount of caffeine compared to mate' plant materials or a lower amount of tannins compared to the kava and mate' plant materials. Compositions of the present invention comprise caffeine amounts that are lower than or equal to the amount of theobromine present in the unextracted mate' plant material, and also comprise compositions comprising a predetermined amount of caffeine wherein the amount of caffeine is lower than or equal to the theobromine amount.

The compositions of the present invention comprise kava compositions having altered kavalactone profiles combined with mate' compositions that have a predetermined characteristic, such as an alkaloid amount, that is unlike that found in the unextracted native plant material and in currently known extracted compositions, such as beverage infusions and decaffeinated products. Compositions having differing predetermined alkaloid amounts allow for the production of kava compositions combined with mate' compositions having differing alkaloid amounts for enhancing or reducing certain physiological effects when the compositions are consumed. Methods of the present invention comprise providing physiological effects including treatments for mental and physical fatigue, headache, diuresis, inflammation, weight reduction, depression, and pain as well as providing improved cognition and mental focus. Embodiments of the compositions provide compositions comprising kava compositions combined with mate' compositions having a caffeine amount that is lower than or equal to the amount of theobromine, and compositions comprising kava compositions combined with mate' compositions having a lowered amount of tannins compared to the kava and mate' native plant materials. Accordingly, an aspect of the present invention relates to methods of selective extraction of the tannin compounds of mate' thereby reducing the risk of oral, esophageal, gastric and bladder cancers associated with excessive kava or mate' consumption.

Another aspect of the present invention relates to formulation of oral delivery systems having the desired clinical effects of enhancing memory, cognition, and sense of well being as well as treatments for obesity and mental or physical fatigue. The compositions of the present invention can be used to make a combined kava extract and mate' extract or theobromine composition product in formulations such as a paste, resin, oil or powder, beverage, liquid infusion or decoction, or a dry flowable powder. Such products are processed for many different uses, and some embodiments are made into a fast-dissolve tablet or other orally available delivery vehicle. The kava or mate' plant material is extracted and the resulting kava or mate' compositions from the extractions of each, have altered kavalactone profiles or predetermined alkaloid amounts and can be in the form of a paste, oil or resin, or other form suitable for use or further processing. Preferably, the extraction methods include using supercritical CO₂ extraction and solvent modifiers such as water and ethyl alcohol. The extracted compositions, having altered kavalactone profiles or predetermined alkaloid amounts, can then be subjected to further processing steps. Mate' and kava compositions produced by such methods have predetermined characteristics, such as altered kavalactone profiles or alkaloid ratios or profiles, that are unlike those found in the native plant materials and the altered kavalactone profile or alkaloid profile can be tailored to meet particular considerations for the final product. The kava and mate' compositions so produced can be used alone or in combination with other compounds or other extracted materials, herbal remedies, pharmaceutical agents, food, dietary supplements, or beverages. The kava and mate' compositions can also be used in treatments of physiological conditions.

### DETAILED DESCRIPTION

The present invention comprises methods and compositions of Piper methysticum or kava, particularly the root material of kava, and mate' or theobromine. Methods of the present invention comprise making compositions comprising extracted kava compositions, which include both the materials extracted from kava and the extracted residue, combined with extracted mate' compositions, or kava extract compositions and theobromine. Compositions of the present invention comprise compositions resulting from extraction of kava, such as compositions of extracted kava that have ratios of alkaloid compounds that are not found in the native plant material, in combination with mate' extract compositions. Suitable methods and compositions of kava and mate' are disclosed in U.S. patent applications Ser. Nos. 10/273,943, 10/408,900, 10/273,981, 10/263,579 and PCT/US03/25819, PCT/US03/31611, PCT/US02/33384 and PCT/US02/31771, PCT/US02/33385, and U.S. Provisional Patent Application No. 60/514,187, and U.S. and international applications which claim the priority of U.S. Provisional Patent Application No. 60/514,187, and the disclosures of each are hereby incorporated by reference in its entirety as if specifically set forth herein.

The kava extract compositions of the present invention are extracts of the native plant material of Piper methysticum, particularly the root material, though other P. methysticum plant material can be used to yield the compositions taught herein. Kava extract compositions comprise one or more kavalactones. Kavalactones contemplated by the present invention include, but are not limited to, methysticin, dihydromethysticin, kavain, dihydrokavain, yangonin, and demethoxyyangonin. The kavalactones in the compositions of the present invention have ratios of one to another that are different from those found in native plant material, and are referred to herein as altered kavalactone profiles. Individual kavalactones are present in various percentages of the total kavalactone component, making up different kavalactone distribution profiles.

The present invention comprises compositions comprising extracts of the plant Piper methysticum or kava combined with compositions comprising extracts of mate', or extracts of kava in combination with theobromine. Another aspect of the present invention comprises methods of use of compositions comprising combinations of kava compositions and mate' or theobromine compositions for the enhancement of memory, cognition, and a sense of well being, as well as for the treatment of fatigue and appetite suppression. More specifically, the present invention comprises methods and compositions of combinations of kava compositions having alkaloid profiles not found in the native plant material and compositions of mate' comprising altered alkaloid profiles. The present invention also comprises methods and compositions of combinations of kava compositions having ratios of compounds such as kavalactones not found in the native plant and mate' compositions, such combination compositions are substantially devoid of tannins or caffeine. As used herein, the termn “kavalactone profile” or “alkaloid profile” shall mean the ratios of kavalactone or alkaloid compounds found in either kava or mate', and the relative amounts of each compound in relation to the other kavalactone or alkaloid compounds in that plant material. The kavalactone profile or alkaloid profile refers to the amount in grams of each kavalactone or alkaloid compound found in kava or mate'. The native plant materials, which have not undergone extractions to remove any components, would have a kavalactone profile or an alkaloid profile exhibiting the types and amounts of kavalactones or alkaloid compounds made by the respective plant. Native plant materials include plant materials that may be shredded, ground or powdered after picking and drying, but no extractions, other than incidental water or oil loss, due to the physical manipulation of the plant material, are included. An altered kavalactone or altered alkaloid profile, or a kavalactone profile or an alkaloid profile different from that of native plant material means the ratios of the kavalactones or alkaloid compounds of the composition are different from the ratios found in the native plant material. For example, in an altered alkaloid profile, the amount of one or more alkaloid compounds may be different or the ratios of one or more alkaloid compounds to the total amount or to other alkaloid compounds are different from that found in native plant material, and the same is true for kavalactones.

Compositions of the present invention comprise compositions of kava extracts, combined with compositions of mate' extracts, or kava extract compositions in combination with theobromine, in formulations such as a paste, powder, or in other forms, for use in dietary supplements. The compositions can be processed to produce consumable items, for example, by mixing it in a food product or in a capsule, or providing the paste itself for use as a dietary supplement, with sweeteners and flavors added as appropriate. Accordingly, such supplements may include, but are not limited to, compositions of kava combined with mate' or theobromine compositions for oral delivery in the form of tablets, capsules, lozenges, liquids, and emulsions. A dry, flowable powder formulation is also contemplated by the present invention. Other aspects of compositions of the present invention comprise kava compositions combined with mate' extract compositions, or theobromine, in the form of a rapid-dissolve tablet.

The kava extract compositions of the present invention are obtained by extraction of the kava components from the root of the plant. In a kava extraction process, the rate at which various components, including but not limited to the kavalactone components, are extracted varies according to the extraction solvent and extraction conditions used. However, kava root extracts are conventionally sold based upon total kavalactone content. Hence, the focus of conventional commercial extraction techniques has been to extract as much of the kavalactone content from the root source as possible to provide for the greatest total kavalactone concentration.

Although various extraction techniques have been used to process kava root, supercritical CO₂ processes have been found to extract the largest quantity of kavalactones. Thus, conventional practice has been to focus on the use of supercritical CO₂ to extract all of the kavalactones from the root in the production of kava extracts. As can be appreciated, such bulk extracts will generally contain kavalactones in the same or nearly the same proportions as present in the source root. Because of the differing physiological effects produced by variations in the kavalactone distribution profile, producers of such kava products are limited to specific kava cultivars as source materials in order to produce a kava product that produces the desired effect.

To the extent that the distribution profile of the various kavalactones in the extract have been of concern, conventional practice is to use additional processing steps, such as high pressure liquid chromatography (HPLC), to extract individual kavalactones. For example, and not for limitation, HPLC and other chromatographic techniques, such as “flash” chromatography, have been used to bulk process kava extracts and isolate individual kavalactones. These latter processes are cumbersome to implement and require the use of machinery that is bulky and prohibitively expensive to operate. Methods for extracting and purifying kava, including supercritical CO₂ and chromatography, are discussed in PCT publication WO 00/772861 to Martin et al.

More recently, methods provided methods for processing kava root to provide an extract of kava as a paste, powder, or in other forms, and to allow the kavalactone profile to be altered during processing. Such methods allow for reduced levels or amounts of methysticin and dihydromethysticin and increased levels or amounts of kavain and dihydrokavain. Such methods are taught in PCT/US2003/25819, PCT/US2002/33385, PCT/US2003/31611, PCT/US2002/333384, and PCT/US2002/31771 to Gow et al, all of which are incorporated herein in their entireties. Such methods can be used to make kava compositions having altered kavalactone profile compositions and that can be produced cost-effectively using a minimal number of processing steps. Additionally, the methods of Gow et al. eliminate the need for additional processing, such as chromatography. For example, and not for limitation, the kava extract compositions of the present invention contain one or more kavalactones in an amount between 0.1 mg to 400 mg. More particularly, the one or more kavalactones present in a kava extract composition range between 1 mg to 300 mg. A specific kava extract composition comprises a total kavalactone amount of between approximately 5 mg to 250 mg.

Of the total kavalactone component of the compositions of the present invention, the percentages of individual kavalactones may vary with respect to one another. For example, and not for limitation, the combined amount of methysticin and dihydromethysticin components, by mass percentage of the total kavalactones present, is less than 30%. More particularly, the combined amount of methysticin and dihydromethysticin components, by mass percentage of the total kavalactones present, may be less than 10%-20%. Within these percentages, the ratio of methysticin to dihydromethysticin may range from 1:10 to 10:1, more particularly from 1:5 to 5:1, and even more particularly from 1:2 to 2:1. Ratios of 1:1 methysticin to dihydromethysticin are specifically contemplated by the present invention.

Also for example, and not for limitation, the combined amount of kavain and dihydrokavain components by mass percentage of the total kavalactone composition is greater than 50%. More particularly, the combined amount of kavain and dihydrokavain components by mass percentage of the total kavalactone composition may be greater than 60%-80%. Within these percentages, the ratio of kavain to dihydrokavain may range from 1:10 to 10:1, more particularly from 1:5 to 5:1, and even more particularly from 1:2 to 2:1. Ratios of 1:1 kavain to dihydrokavain are specifically contemplated by the present invention.

Also for example, and not for limitation, the combined amount of yangonin and demethoxyyangonin components by mass percentage of the total kavalactone composition is less than 20%. As used herein, demethoxyyangonin means 2H-Pyran-2-one, 4-methoxy-6-styryl-, (E)-, CAS no. 15345-89-8, and is also known as 2H-Pyran-2-one, 4-methoxy-6-styryl-,(E)-(E)-4-Methoxy-6-(2-phenylethenyl)-2H-pyran-2-one;2H-Pyran-2-one, 4-methoxy-6-(2-phenylethenyl)-,(E)-;4-Methoxy-6-styryl-2H-pyran-2-one; Demethoxyyangonin; desmethoxyyangonin; or DMY. More particularly, the combined amount of yangonin and demethoxyyangonin components by mass percentage of the total kavalactone composition may be less than 5%-15%. Within these percentages, the ratio of yangonin and demethoxyyangonin may range from 1:10 to 10:1, more particularly from 1:5 to 5:1, and even more particularly from 1:2 to 2:1. Ratios of 1:1 yangonin and demethoxyyangonin are specifically contemplated by the present invention.

One embodiment of the present invention includes compositions wherein the kava extract composition comprises combined kavalactones in an amount between 0.1 mg to 400 mg wherein the combined amount of methysticin and dihydromethysticin components, by mass percentage of the total kavalactones present, is less than 30%, the combined amount of kavain and dihydrokavain components by mass percentage of the total kavalactone composition is greater than 50%, and the combined amount of yangonin and demethoxyyangonin components by mass percentage of the total kavalactone composition is less than 20%.

Kava extract compositions of the present invention comprise reduced levels of methysticin and dihydromethysticin and increased levels of kavain and dihydrokavain. An embodiment of the present invention comprises a kava extract composition having a kavalactone profile similar to that found in extracts of “one-day” cultivars from Vanuatu made from kava feedstock from a less desired cultivar of Piper methysticum. Compositions having lower amounts of methysticin and dihydromethysticin and higher amounts of kavain and dihydrokavain are contemplated by the present invention. Kava extract compositions of the present invention have kavalactone profiles that are unlike those found in natural kava plant material, an altered kavalactone profile, and the profile can be tailored to meet particular end product considerations. The native kava plant material, referred to herein interchangeably as kava or Piper methysticum, has not undergone extractions to remove any components, and has a kavalactone profile exhibiting the types and amounts of kavalactone compounds made by the plant.

Embodiments of the kava extract compositions of the present invention have kavalactone amounts that have been selected to be low in the combined weight percentage of methysticin (M) and dihydromethysticin (DHM), low in the combined weight percentage of desmethoxy yangonin (DMY) and yangonin (Y), and high in the combined weight percentage of kavain (K) and dihydrokavain (DHK). Kava extract compositions can be characterized according to one or more of the following attributes: (a) a combined weight percentage of six major kavalactones methysticin (M), dihydromethysticin (DHM), yangonin (Y), desmethoxyyangonin (DMY), kavain (K), and dihydrokavain (DHK) of between about 20% to a maximum of about 90%; (b) a combined weight percentage of M and DHM between about 5-15% to about 29%; (c) a ratio of Y to DMY by weight, expressed as the logarithmic function 10*LOG₁₀(Y/DMY) in dB units, from about −1 to about 2; (d) a ratio of DHK to K by weight, expressed as the logarithmic function 10*LOG₁₀(DHK/K) in dB units, from about −4 to about 1; (e) a combined weight percentage of Y and DMY between about 5% to about 20-25%, and (f) a combined weight percentage of flavokavain A and flavokavain B of between about 0.3% to about 3%.

Embodiments (c) and (d) refer to mathematical representations of relationships between the amount of Y to DMY, and DHK to K, respectively. The data is represented in dB units, and the calculation is 10 times the log value of Y% divided by DMY%, or DHK% divided by K%. For example, if the yangonin percent is 10%, and the demthoxyyangonin percent is 16%, then 10% divided b 16% equals 0.625, and the log of 0.625 is −0.204. 10 times −0.204 equals −2.04dB. Such calculations can be easily represented in graphical fashion, as shown by the figures in PCT/US02/31771, incorporated herein by reference in its entirety.

The kava extract compositions of the present invention also comprise other combinations of kavalactones. In accordance with the present invention, compositions of kava extract compositions can comprise compositions comprising altered kavalactone profiles comprising combined weight percentage of the six major alpha-pyrones: methysticin (M), dihydromethysticin (DHM), yangonin (Y), desmethoxyyangonin (DMY), kavain (K), and dihydrokavain (DHK); combined weight percentage of M and DHM; combined weight percentage of DHK and K; combined weight percentage of Y and DMY; ratio of the weight percentages of Y and DMY, i.e., Y/DMY; ratio of the weight percentages of DHK and K, i.e., DHKIK; and combined weight percentages of flavokavain A and flavokavain B.

An embodiment of a kava extract composition, provided in for example, a kava paste or a dry flowable power for use in oral delivery, has one or more of the following properties: total weight percentage of M+DHM+Y+DMY+DHK+K in a range of from about 20% to about 90%; combined M+DHM weight percentage ranging from about 15% or lower to a maximum of about 29%; combined weight percentage of DHK and K in a range from about 50% to about 70%-80%; combined weight percentage of Y and DMY ranging from about 5% to about 25%; ratio of Y weight percentage to DMY weight percentage, expressed as the logarithmic function 10*LOG in dB units, ranging from about −1 to a maximum of about 2; ratio of DHK weight percentage to K weight percentage, expressed as the logarithmic function 10*LOG in dB units, ranges from about −4 to about 1; and combined weight percentages of flavokavain A and flavokavain B ranges from about 0.3% to about 3%. The value of these properties in a kava extract composition can be determined using conventional analytical techniques, such as HIPLC-UV-S (High Performance Liquid Chromotography with Ultra-Violet detection (254 nm) and Chemical Standards), or HPLC Electrospray-Mass Spectrometry.

One embodiment of a kava extract composition comprises less than about 15% of the combination of methysticin and dehyromethysticin, less than about 8% of the combination of yangonin and desmethooxyyangonin, and greater than about 70% of the combination of kavain and dihydrokavain. Another embodiment comprises up to about 7% methysticin, up to about 5% dehyromethysticin, up to about 1 % yangonin, up to about 4% desmoxyyangonin, and greater than about 38% kavain.

For a person skilled in the art, the kavalactones can be extracted from Piper methysticum, preferably the root, via a variety of different means including commonly used liquid extraction methods, including liquid extraction at atmospheric pressure. Liquid extraction may be performed by any means known in the art and include solvents such as, but not limited to, ethyl alcohol, acetone, or dimethyl ether. Alternatively, liquid extraction at atmospheric pressure may be used to extract the active kava compounds from the root source, wherein compressible gases such as, but not limited to, carbon dioxide, propane, or tetrafluoroethane may be used. When using the liquid solvents at atmospheric pressure, an apparatus such as a Soxhlet extractor will efficiently extract from the root the desired resinous material, and, thereafter, the solvent can be removed via a distillation step for later reuse, leaving behind the desired kavalactones contained in a matrix of resinous material.

In a similar fashion, kava extract can be obtained from the kava root using compressible gases such as carbon dioxide in the liquid or supercritical state, or propane in the liquid state. In this method of preparation/collection of the essential oil, the kava root material is contained within a vessel that can be pressurized and the chosen liquid (compressed gas) is passed through the vessel under pressure. In the case of carbon dioxide, the pressure ranges from 1500 psi to over 5000 psi, and in the case of the other liquefied gases, the pressure is an order of magnitude less ranging from 50 psi to 400 psi, while in both cases the temperature can range from 5 ° C. to 60 ° C. To recover the resinous material within which contains the kavalactones, the extract-laden liquid is then passed through a collection vessel, wherein the liquefied gas can be collected as a vapor, leaving behind the desired kavalactones that were originally contained in the root material. Subsequently, the gas is compressed to a liquid for reuse. An improved kavalactone profile can then be achieved by performing an additional extraction step comprising use of supercritical CO₂ as a mobile phase, combined with an adsorbent material such as that used for solid-phase extractions (SPE) and/or diatomaceous earth. Alternatively, the the kavalactones, including but not limited to methysticin, dihydromethysticin, kavain, dihydrokavain, yangonin, and demethoxyyangonin, can be purchased individually from a chemical supply company such as Chromadex and mixed in the desired proportions.

Another method to obtain different kavalactone profiles is to use the root of different varieties of the kava plant or different parts of the kava plant as initial feedstock for purposes of extraction. Alternatively, chromatographic methods as described above can be used to obtain purified forms of each of the kavalactones.

The present invention also comprises methods and compositions of mate' or theobromine combined with one or more of the kava extract compositions described herein. As used herein, mate' refers to the plant or plant material derived from the plant Aquifoliaceae, Ilex genus, wherein the genus includes but is not limited to, I. paraguariensis, I. theezans C. Martis ex Reisseck, I. dumosa Reisseck; I. dumosa Reisseck var dumosa; I. argentina Lillo; I. brevicuspis Reisseck; I. microdonta Reisseck; I. paraguariensis St. Hil. var. paraguaraniensis; I. paraguariensis St. Hil. var. vestita (Reiss.); and I. pseudobuxus Reisseck. The term also includes all clones, cultivars, variants, and sports of Ilex. The term “Ilex” is also used interchangeably with “mate'” and means these plants, clones, variants, sports, etc. As used herein, when the tea-like beverage made from this plant genus is referred to, the beverage is designated as “mate' beverage”. Compositions of the present invention preferably comprise extracts of the leaf of I. paraguariensis.

The compositions of the present invention are useful in providing the physiological effects of enhanced memory, improved cognition, reduced mental and physical fatigue, a sense of well being, and appetite suppression. Though each plant material, kava and mate', have been consumed by humans for some of these effects, the present invention provides compositions that are free from carcinogens such as the tannin compounds, and also provide effects different from the natural plant materials, for example, due to the lack of caffeine.

Principal bioactive chemical constituents of mate' are listed in Table 1. Though not wishing to be bound by any particular theory, it is currently believed that beneficial effects of aqueous mate' extracts include protecting low density lipoprotein (LDL) from oxidative damage and can inhibit the atherosclerotic process. The mate' effect (18.5 mM) has been found to be greater than that measured for red wine (0.74 mM). Mate' extracts have been shown in in vitro assays to inhibit peroxidation in a concentration dependent manner which should protect cell membrane lipids as well as having a red blood cell protectant effect against hydrogen peroxide generated free radicals. It is thought that the antioxidant activity is due to caffeoyl derivatives. Mate' may also play a protective role in the process of glycation. Glycation has been proposed as a key to diabetic complications resulting from hyperglycemia. The chlorogenic acids of mate' have been demonstrated to be potent and selective inhibitors of HIV integrase and the polyphenols have been shown to inhibit formation and growth of neoplasms. Mate' has also been demonstrated to possess conentation dependent vasorelaxing activity, diuretic effects, bronchial smooth muscle relaxation and reduction in appetite with increase in liver metabolic processes.

TABLE 1
Principal Bioactive Chemicals of I. paraguariensis

| Constituents | % Dried Weight |
|---|---|
| Methylxanthines | |
| Caffeine | 0.5-2.2 |
| Theobromine | 0.03-0.6 |
| Theophylline | 0.004-0.08 |
| Caffeoyl Derivatives | 9.0-11.0 |
| Saponin Glycosides | 5.0-10.0 |
| Tannins | 7.0-14.0 |

Compositions of the present invention comprise extracts of kava and mate'. Such compositions have differing amounts of alkaloids, kavalactones, and tannin compounds, than those found in the native plant materials. An aspect of the present invention comprises compositions having lowered amounts of methysticin and dihydromethysticin, and increased amounts of kavain and dihydrokavain in comparison to the levels found in native kava plant materials. Another aspect of the present invention comprises compositions of kava extract compositions with substantially no tannins and mate' extract compositions with substantially no tannins. Yet another aspect of the present invention comprises compositions of mate' extracts having a lowered caffeine concentration and substantially no tannins in relation to the concentrations found in the native mate' plant material. Compositions also comprise kava extract compositions combined with theobromine.

Compositions of the present invention comprise mate' extracts of the leaf of I. paraguariensis. An aspect of the present invention comprises compositions of mate' extracts of the leaf of I. paraguariensis having a lowered concentration or amount of caffeine in relation to the concentration or amount found in the native plant material. Another aspect of the present invention comprises compositions of mate' extracts of the leaf of I. paraguariensis having reduced or substantially no tannin compounds in relation to the concentration or amounts found in the native plant material.

The present invention comprises compositions of mate' extracts, and compositions of theobromine. Theobromine may be derived from mate', other sources, or be made by synthetic means known to those skilled in the art. Purine alkaloids, also referred to herein as methylxanthines, such as caffeine (1,3,7-trimethyl-xanthine), theobromine (3, 7-dimethyl-xanthine) and theophylline (1,3-dimethyl-xanthine) are synthesized in many higher plants. 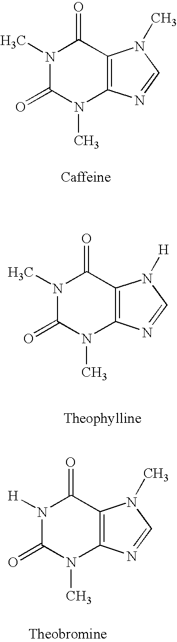
These three alkaloids, along with other methylxanthines, vitamins, minerals, fats, carbohydrates, proteins, nucleic acids and other plant cell constituents, are found in mate' plant material. In the mate' plant, the amounts of each alkaloid and the ratios of one to the others is variable and is dependent upon such factors as genetic variability, environmental conditions, harvest period, and other factors that influence the growth of plants. Additionally, the industrial processing methods used to make commercial products from mate' plants cause further changes in the chemical constituency of the products. As used herein, the term “mate' constituents” shall mean chemical compounds found in mate' and shall include all such chemical compounds identified above as well as all other compounds found in mate'. The native mate' plant material has variable and unknown amounts of such alkaloid compounds, and the processing of the plant material introduces more variability in the amounts of alkaloid compounds found in the products that are consumed. This increased variability in the products consumed leads to widely fluctuating physiological changes in humans and animals ingesting such products, hinders effective treatments using mate' products or prevents avoidance of unwanted physiological effects from ingestion of mate' products.

The present invention comprises compositions comprising combinations of mate' compositions and kava compositions, wherein at least one of the compositions, for example either the mate' composition or the kava composition, has a different compound profile, or different compound amounts, than the native plant material. Further, compositions of the present invention may comprise at least one mate' or kava compositions that has the same compound profile or same compound amounts as are found in the native plant material. For example, a composition of the present invention may comprise a kava extract composition with the altered profile or altered amounts of one or more compound of any of those taught or suggested herein in combination with a mate' composition that has the profile or compound amounts of native mate' plant material. The mate' compositions range from compositions of mate' plant material that has not undergone any extraction of compounds, other than drying the plant material, to mate' extract compositions that have undergone one or more extraction steps taught herein. Also for example, a composition of the present invention may comprise a mate' extract composition with the altered profile or altered amounts of one or more compounds of any of those taught or suggested herein in combination with a kava composition that has the profile or compound amounts of native kava plant material that has been picked and dried. The kava compositions range from compositions of kava plant material that has not undergone any extraction of compounds, other than drying the plant material, to kava extract compositions that have undergone one or more extraction steps taught herein. The compositions of the present invention also comprise the combination of one or more of the kava compositions with one or more of the mate' compositions with theobromine. Such combinations include all three types of compositions, kava, mate' and theobromine, or combinations of any two types of compositions, kava with mate', kava with theobromine, mate' with theobromine.

Compositions of the present invention comprise extracts of kava, combined with extracts of mate', or theobromine, as a paste, powder, or in other forms, which allows the compounds in the extract, such as alkaloids, to be used in dietary supplements. The extracts can be processed to produce such consumable items, for example, by mixing it in a food product or in a capsule, or providing the paste itself for use as a dietary supplement, with sweeteners and flavors added as appropriate. Accordingly, such supplements may include, but are not limited to, compositions of Kava extracts combined with mate' extract compositions, or theobromine for oral delivery in the form of tablets, capsules, lozenges, liquids, and emulsions. Other aspects of compositions of the present invention comprise kava extracts combined with mate' extract compositions or theobromine in the form of a rapid-dissolve tablet.

Compositions of the present invention comprise combinations of the compositions of kava with compositions of mate' or kava extract compositions combined with theobromine. Compositions of the present invention comprise one or more compounds of kava extracts in combination with one or more compounds of mate' extract compositions. The compositions of kava extracts or the mate' extract compositions may have one or more of the altered alkaloid profiles or altered kavalactone profiles taught herein. As used herein, the term “one or more compounds” means that at least one compound, such as kavain or theobromine is intended, or that more than one compound, for example kavain and dihydrokavain, or theobromine and theophylline is intended. As is known in the art, the term “compound” does not mean one molecule, but multiples or moles of molecules of one or more compounds.

The present invention comprises compositions comprising a combination of one or more compounds found in mate' with extracts of kava, comprising kavalactones in concentrations that are different from those concentrations found in native kava plant material, or with extracts wherein the amounts of methysticin and dihydromethysticin are less than the amounts of kavain and dihydrokavain. The present invention also comprises ingestible products that comprise the compositions comprising kava extracts and mate' extract compositions taught herein. For example, the present invention comprises compositions comprising a rapid dissolve tablet, comprising an kava extract composition having an kavalactone profile wherein the kavain and dihydrokavain compounds are in a higher concentration than methysticin and dihydromethysticin compounds and a mate' extract composition wherein the caffeine has been removed or reduced to amounts lower than the amounts of theobromine, and wherein the amount of tannin compounds is reduced or substantially no tannins are present. In another embodiment of the present invention, compositions comprise mate' extract compositions in combination with kava extract compositions. The kava extract compositions may comprise different ratios of kavalactones. Kava extract compositions having 0% methysticin and 100% kavain can be combined with the mate' extract compositions.

The present invention comprises compositions and methods for making and using such combination kava extract and mate' extract compositions, where the compositions comprise oral delivery dosage formulations comprising the compositions taught herein. An aspect of the present invention comprises a rapid dissolve tablet, comprising a composition of mate' extract composition in combination with a kava extract composition, or theobromine combined with a kava extract composition, wherein the kava extract composition has an altered kavalactone profile.

Each and every one of the kava compositions disclosed herein can be combined with compositions of mate' extracts or with theobromine alone. Accordingly, an aspect of the present invention comprises extractions of mate' for combining with the kava extraction compositions described herein. Steam distillation techniques that are known to those skilled in the art may be used obtain extracts of mate' from the mate' plant material. The mate' plant material may be the aerial portion of the plant, which includes the leaves, stems, flowers, branches, twig and trunk, or other plant parts, though leaves and stems are preferred starting material. The extract can be obtained from the mate' leaves via the process of steam distillation of the leaves or by liquid extraction techniques such as using dichloromethane or petroleum ether as the extracting solvent. Alternatively, an extract of the dried leaf material can be prepared using carbon dioxide in the liquid or supercritical phase, or, a liquefied gas such as tetrafluoroethane or propane. In the case of carbon dioxide, the pressure ranges from about 1500 psi to over about 5000 psi, and in the case of the other liquefied gasses the pressure is an order of magnitude less ranging from about 50 psi to about 400 psi. The extract-laden liquid is then passed through a collection vessel wherein the liquefied gas can be collected as a vapor leaving behind the desired extract that was contained in the leaf. Although the mate' extract may be obtained from any species of from the Ilex genus, the extract is preferably obtained from I. paraguariensis.

Alternatively, the chemical constituents found within mate', such as, but not limited to, caffeine or theobromine, can be purchased individually from a chemical supply company. For example, purified theobromine that has been extracted from a natural source, such as Cacoa, may be obtained commercially from the company Natra. Chemically synthesized theobromine can also be obtained from many different chemical supply companies such as Sigma Aldrich. The individual chemical constituents found in mate' may be purchased and combined with the kava extract compositions described herein. Such chemical constituents may also be mixed in the proportions that exist in mate' prior to combining with the kava extract compositions described herein.

The present invention comprises methods for producing compositions of mate' extracts that have predetermined characteristics, including but not limited to, predetermined concentrations or amounts of alkaloid compounds. Embodiments comprise compositions of mate' having a caffeine concentration that is less than or equal to the theobromine concentration in the mate' composition. The amounts of methylxanthines present in mate' are generally known. The amounts of methylxanthines found in the leaves of Ilex paraguariensis, on a dry weight basis, range from approximately 0.002% to 0.015% theophylline; 0.30% to 0.60% theobromine; and 0.80% to 2.00% caffeine. Compositions of the present invention comprise extracted mate' compositions having predetermined caffeine concentrations, that when compared on an equal weight basis to the native plant material equal to or approximately less than 0.60%, approximately less than 0.55%; approximately less than 0.50%; approximately less than 0.45%; approximately less than 0.40%; approximately less than 0.35%; approximately less than 0.30%; approximately less than 0.25%; approximately less than 0.20%; approximately less than 0.15%; approximately less than 0.10%; in the mate' extract compositions the amount of caffeine can include all ranges from 0% to less than or equal to the concentration or amount of theobromine in the mate' composition.

Methods for producing such compositions comprise extraction of mate' plant material to alter the amount of one or more compounds from an amount or amounts found in the native plant material, preferably such compounds comprise alkaloid compounds, and most preferably, such compounds comprise caffeine. These compositions include both the extract product resulting from extractions methods and the residue from the extraction, including plant material that was extracted and intermediary extracted residues from subsequent extractions.

The native mate' plant material may undergo pre-extraction steps to render the material into a form more easily extracted, though that form is not limited to any particular form, and any form that is useful for extraction is contemplated by the present invention. Such pre-extraction steps include, but are not limited to, wherein the material is chopped, minced, shredded, ground, pulverized, cut, or torn, and the starting material, prior to pre-extraction steps, is dried or fresh plant material. Another pre-extraction step includes soaking the plant material so that the plant material has a prescribed desired water content. A preferred pre-extraction extraction step comprises cutting the mate' leaves into small pieces known as tea cut. The starting material or the pre-extraction material can then be dried or can have moisture added to it. Once the plant material is in a form for extraction, methods of extraction are contemplated by the present invention and are taught in U.S. Provisional Patent Application 60/514,187, and in applications claiming priority to U.S. Provisional Patent Application 60/514,187.

An aspect of the present invention comprises methods for extracting the mate' plant material to remove one or more of the methylxanthines as well as flavor compounds and optionally, other compounds found in mate'. This resulting extraction composition, denoted herein as “an extracted mate' composition” preferably comprising a composition comprising a methylxanthine concentration wherein the concentration of caffeine is less than or equal to the concentration of theobromine, does not comprise the extracted plant material, but only the components, or compounds, extracted from the plant material. The extracted mate' composition, comprising extracted methylxanthines, and/or flavor compounds and other compounds, is formulated with known pharmaceutical agents to provide a pharmaceutical composition. The pharmaceutical composition has an effective amount of one or more of the extracted methylxanthines, preferably having a caffeine concentration that is equal to or less than the theobromine concentration. An aspect of the invention comprises mate' compositions having a lower amount of caffeine in relation to the level found in conventional leaf extracts. Methods of decaffeination have been well documented in the case of coffee. Descriptions of such methods are described in Katz. SN., “Decaffeination of Coffee”, Coffee: Technology, Ed. Clark RJ and Macrae R., New York, Elsevier Applied Science, 1987; and Pintauro ND., Coffee Solubilization: Commericial Process and Techniques, Park Ridge, Noyes Data Corporation, 1975; the teachings of which are incorporated herein by reference as if entirely set forth. As with coffee, the process of decaffeination of the leaves of mate' can be accomplished in a similar fashion.

Another embodiment of the invention comprises mate' compositions having reduced or substantially no tannins in relation to the level found in the native plant material or in mate' beverages. To remove the tannins from the mate' plant material, or from the decaffeinated extract of mate', adsorbents, such as fining with albumin, or activated charcoal, or anion exchange resins are added. For a person skilled in the art, the removal of tannins through the addition of such adsorbents is accomplished in a straightforward manner by using hot water in a manner described as a decoction or infusion. The methods for removing tannins include the addition of adsorbents, such as albumin or activated charcoal, or by anion exchange resins, or others known in the art may be used for removing tannins from the extracts of kava or mate', or both. In order to prepare a finished product for consumption, it is often beneficial to remove the water from the extract that has been prepared. The water may be removed using techniques known to those skilled in the art such as, but not limited to, vacuum distillation, spray drying, refractive window drying, or freeze drying of the product having reduced caffeine levels and substantially no tannins in relation to the levels found in the leaf material.

Other compositions of the present invention comprise extracted mate' plant materials. Embodiments of extracted plant materials comprise mate' that has undergone extraction methods described herein to remove compounds so that the extracted plant material has a predetermined characteristic, such as a predetermined alkaloid profile, particularly a methylxanthine concentration, in the remaining plant materials. An embodiment comprises extracted mate' plant material that comprises a methylxanthine profile wherein the caffeine concentration is less than or equal to the theobromine concentration. As used herein, a mate' extract composition is intended to include the composition comprising extracted mate' plant materials or the composition comprising the extraction composition resulting from extraction of mate' plant material. Either mate' composition can be used in the present invention and the compositions are interchangeable unless otherwise indicated.

In general the mate' plant material that was extracted with supercritical CO₂, having an altered caffeine concentration, is recovered and further extracted with a hydroalcoholic solution in any one of the methods described below. An aspect of the compositions made using these methods is a composition comprising an altered alkaloid profile, and preferably an alkaloid profile wherein the amount of caffeine is less than or equal to the amount of theobromine in the composition.

In one method, extracted mate' leaf material is mixed into a hydroalcoholic solution, which is 50% to 95% ethyl alcohol content in water, and preferably between 75% and 90% ethyl alcohol content, in a ratio of solution to mate' material (liters:kg) ranging from 2:1 to 20:1. The mixture of leaf material and hydroalcoholic liquid is heated from 20° Celsius (C.) to 60° C., and mixed for a period of time of between 1 hour and 12 hours. One method for mixing comprises using a kettle that is jacketed such that the temperature is controlled. The kettle is closed and the mixture is stirred slowly. After the desired time of mixing, the liquid is separated from the solid material by means known to those skilled in the art, including but not limited to, filtration or centrifugation. The remaining solid material may be further extracted one or more times by the above steps of hydroalcoholic solution, heating and mixing to yield extracted mate' compositions that can be used independently or can be pooled with other extracted mate' compositions. Alternatively, the resulting material from the hydroalcoholic extraction methods can undergo supercritical CO₂ extraction, refrigerant extraction or other extractions to yield extracted mate' compositions that can be used independently or pooled with other extracted mate' compositions. An aspect of the compositions made using this method is a composition comprising an altered alkaloid profile, and preferably an alkaloid profile wherein the amount of caffeine is less than or equal to the amount of theobromine in the composition.

A further embodiment of a hydroalcoholic extraction method of the present invention comprises separate solutions of water and alcohol in a Soxhlet or pseudo-Soxhlet extraction process. The Soxhlet extraction process is a well known method for extracting materials. The Soxhlet extraction process or pseudo-Soxhlet extraction process can occur under normal atmospheric or reduced atmospheric pressure. In the Soxhlet extraction process the leaf material is held apart from the reservoir of solvent and a condenser element is above the leaf material onto which the solvent condenses and drips onto, into, and through the leaf material making the extract that collects into the reservoir below. This extraction process can be performed sequentially with water first and alcohol thereafter and then pooling the two individual liquid extracts, or alcohol first, followed by water, and then pooling the extracts. The resulting extracted mate' composition from the Soxhlet extraction methods can undergo further extractions, including but not limited to, supercritical CO₂ extraction, refrigerant extraction or other extractions, to yield extracted mate' compositions. The remaining solid material may also be further extracted one or more times by the Soxhlet extraction methods, or other extractions methods, to yield extracted mate' compositions that can be used independently or pooled with other extracted mate' compositions. An aspect of the compositions made using this method is a composition comprising an altered alkaloid profile, and preferably an alkaloid profile wherein the amount of caffeine is less than or equal to the amount of theobromine in the composition.

In performing the extraction methods above, it was found that the dried bulk hydroalcoholic extract of the leaves of Ilex paraguariensis amounts to between 10% to 30% by weight (excluding the carrier material) of the original dried Ilex paraguariensis leaves used. Using extraction methods such as those disclosed above, the desired alkaloid profiles are created in the mate' extract compositions, whether it is the extracted mate' compositions or in the extracted plant material compositions. Alternatively, the mate' plant material could be extracted to remove one, two or all or almost all of at least three methylxanthines, caffeine, theophylline and theobromine, to produce either an extracted mate' composition substantially free of one or more of these compounds, to produce an extracted plant material composition free of one or more of these compounds, or to produce a composition comprised of at least one, two or three of methylxanthines. The specific extraction environments, rates of extraction, and solvent used depends on the starting profile of the source material and the degree of profile change desired. Specific solvent and environmental attributes can be determined by those of ordinary skill in the art using no more than routine experimentation typical for adjusting a process to account for, e.g., variations in the attributes of starting materials that is to be processed to produce an output material that has specified attributes. For example, in a particular lot of mate' plant material, the initial concentrations of caffeine, theobromine and theophylline are determined using methods known to those skilled in the art, such as by extraction and measurement of each using chromatography such as high performance liquid chromatography. One skilled in the art can determine the amount of change from the initial concentrations of methylxanthines to the predetermined amounts of methylxanthines for the final product and the extraction methods, as disclosed herein, to reach the desired profile of the final mate' compositions. See Table 2.

TABLE 2
Mate' Constituents (% dry weight)

| | Caffeine | Theobromine | Theophylline | Tannins |
|---|---|---|---|---|
| Mate' Feedstock | 2.0 | 0.5 | 0.1 | 12.0 |
| Extract | 0.8 | 1.1 | 0.2 | 8.0 |
| Post-Supercritical CO₂ | | | | |
| Extract | 1.0 | 1.3 | 0.3 | 1.0 |
| Post-Fining (albumin) | | | | |

An embodiment of a composition comprises a mate' extract composition having a predetermined caffeine concentration that is less than or equal to the original theobromine concentration that is found in the native plant material, or a predetermined theobromine concentration such as that which can result from extraction techniques taught herein, and comprises substantially no tannin compounds.

The mate' extract composition, having a predetermined alkaloid profile and substantially no tannin compounds, can be processed to produce consumable items, for example, by mixing it in a food product or in a capsule, or providing the extracted mate' plant material itself or an extracted mate' composition for use as a dietary supplement, or beverage with sweeteners and flavors added as appropriate. According to a further aspect of the invention, the mate' extract composition can be further processed to produce a dry, flowable powder. The powder can be used as a dietary supplement that can be added to various edible products. The powder or the final predetermined unique extract of mate' is also suited for use in a rapid dissolve tablet.

According to a particular aspect of the invention, the mate' extract composition is produced to have a predetermined alkaloid profile, preferably having a caffeine concentration less than or equal to the concentration of theobromine, that is particularly well suited for delivery in the oral cavity of human subjects, e.g., via a rapid dissolve tablet. Additionally, the mate' extract composition may or may not have substantially no tannin compounds present.

Once a dry powder, comprising kava extract compositions, mate' extract compositions, theobromine compositions, or combinations of two or more of these compositions, is obtained, it can be used in a variety of ways such as a dietary supplement, for tableting for addition to food substances or for other uses. In a particular embodiment, the powder is mixed with other ingredients to form a tableting composition of powder which can then be formed into tablets. In a particular embodiment, the tableting powder is first wet with a solvent comprising alcohol, alcohol and water, or other suitable solvents, in an amount sufficient to form a thick doughy consistency. Suitable alcohols include, but are not limited to, ethyl alcohol, isopropyl alcohol, denatured ethyl alcohol containing isopropyl alcohol, acetone, and denatured ethyl alcohol containing acetone. The resulting paste is then pressed into a tablet mold. An automated molding system, such as described in U.S. Pat. No. 5,407,339 can be used. The tablets are then removed from the mold and dried, preferably by air-drying for at least several hours at a temperature high enough to drive off the solvent used to wet the tableting powder mixture, typically between about 70° C. to about 85° F. The tablets can then be packaged for distribution.

The kava extract compositions and mate' extract compositions or theobromine can be combined using techniques and methods that are known in the art. Such techniques include, but are not limited to, mixing, blending, stirring, including mechanical stirring, and dissolving.

Methods and compositions of the present invention comprise compositions comprising combinations of compositions of kava and mate' or theobromine in the form of a paste, resin, oil, or powder. An aspect of the present invention comprises compositions of liquid preparations of kava extract compositions combined with liquid preparations of mate' extract compositions or theobromine. Liquid preparations for oral administration may take the form of, for example, solutions, syrups or suspensions, or they may be presented as a dry product for reconstitution with water or other suitable vehicles prior to administration. Such liquid preparations may be prepared by conventional means with pharmaceutically acceptable additives such as suspending agents (e.g. sorbitol syrup, methyl cellulose, or hydrogenated edible fats); emulsifying agents (e.g. lecithin or acacia); non-aqueous vehicles (e.g. almond oil, oily esters or ethyl alcohol); preservatives (e.g. methyl or propyl p-hydroxybenzoates or sorbic acid); and artificial or natural colors and/or sweeteners. Compositions of the liquid preparations can be administered to humans or animals in pharmaceutical carriers known to those skilled in the art. Such pharmaceutical carriers include, but are not limited to, capsules, lozenges, syrups, sprays, rinses, and mouthwash.

An aspect of the present invention comprises compositions of a dry powder extract of kava combined with a dry powder extract of mate' or a dry powder comprising theobromine. Such dry powder compositions may be prepared according to methods disclosed herein and by other methods known to those skilled in the art, such as, but not limited to, spray air drying, freeze drying, vacuum drying, and refractive window drying. The combined dry powder compositions can be incorporated into a pharmaceutical carrier such as, but not limited to, tablets or capsules, or reconstituted in a beverage such as a tea.

Although the extraction techniques described herein are discussed in terms of kava and mate', it should be recognized that compositions of the present invention can also comprise, in the form of a dry flowable powder or other forms, extracts from other plants such as, but not limited to, varieties of ginseng, cherry, lettuce, Echinacia, piper betel leaf, muira puama, ginger, willow, suma, damiana, areca, horny goat weed, ginkgo biloba, turmeric, garlic, puncture vine, arctic root astragalus, eucommia, gastrodia, and uncaria, or pharmaceutical or nutriceutical agents.

The present invention comprises compositions comprising combinations of kava extract compositions and mate' extract compositions or theobromine compositions in tablet formulations, and methods for making such tablets. A tableting powder can be formed by combining between about 18% to about 60% by weight of the powdered kava extract composition and about 1% to about 40% by weight of the powdered mate' extract composition, or a theobromine composition, with between about 30% to about 80% by weight of a dry water-dispersible adsorbant such as, but not limited to, magnesium carbonate, or a dilutent, such as, but not limited to, lactose. Other dry tablet additives, such as, but not limited to, one or more of a sweetener, flavoring and/or coloring agents, a binder, such as acacia or gum arabic, a lubricant, a disintegrant, and a buffer, can also be added to the tableting powder. The dry ingredients are screened to a particle size of between about 50 to about 150 mesh. Preferably, the dry ingredients are screened to a particle size of between about 80 to 100 mesh.

A wide variety of tablet formations can be made. Preferably, the tablet has a formulation that results in a rapid dissolution or disintegration in the oral cavity. The tablet is preferably of a homogeneous composition that dissolves or disintegrates rapidly in the oral cavity to release the extract content over a period of about 2 seconds or less to about 60 seconds or more, preferably about 3 to about 45 seconds, and most preferably between about 5 to about 15 seconds.

Various rapid-dissolve tablet formulations known in the art can be used. Representative formulations are disclosed in U.S. Pat. Nos. 5,464,632; 6,106,861; 6,221,392; 5,298,261; 6,221,392; and 6,200,604; the entire contents of each are expressly incorporated by reference herein as if specifically set forth. For example, U.S. Pat. No. 5,298,261 teaches a freeze-drying process. This process involves the use of freezing and then drying under a vacuum to remove water by sublimation. Preferred ingredients include hydroxyethylcellulose, such as Natrosol from Hercules Chemical Company, added to between 0.1% and 1.5%. Additional components include maltodextrin (Maltrin, M-500) at between 1% and 5%. These amounts are solubilized in water and used as a starting mixture to which is added a composition comprising a combination of a kava extract composition and a mate' extract composition or theobromine composition, or individually the kava extract composition and the mate' extract composition or theobromine composition, along with flavors, sweeteners, such as Sucralose or Acesulfame K, and emulsifiers such as BeFlora and BeFloraPlus which are extracts of mung bean.

A particularly preferred tableting composition or powder contains about 10% to about 60% by weight of a kava extract powder and a mate' extract powder, or a theobromine composition, and about 30% to about 60% of a water-soluble diluent. Suitable diluents include lactose, dextrose, sucrose, mannitol, and other similar compositions. Lactose is a preferred diluent but mannitol adds a pleasant, cooling sensation and additional sweetness in the mouth. More than one diluent can be used. A sweetener can also be included, preferably in an amount of between about 3% to about 40% by weight depending on the desired sweetness. Preferred sweetening substances include, but are not limited to, sugar, saccharin, sodium cyclamate, aspartame, and Stevia extract, used singly or in combination, although other sweeteners could alternatively be used. Flavorings, such as mint, cinnamon, citrus (e.g., lemon or orange), can also be included, preferably in an amount between about 0.001% to about 1% by weight. If a coloring is desired, natural and/or synthetic colors can be added, preferably in an amount of between about 0.5% to about 2% by weight.

Typically, this tableting composition will maintain its form without the use of a binder. If needed, however, various binders are suitable and can be added in an amount of between about 5% to about 15% by weight, or as necessary. Any binder known to one of ordinary skill in the art may be used. Preferred binders include, but are not limited to, acacia or gum arabic. Alternative binders include sodium alginate, extract of Irish moss, panwar gum, ghatti gum, mucilage of isapol husks, carboxymethylcellulose, hydroxyethylcellulose, methylcellulose, polyvinylpyrrolidone, VEEGUM.RTM. (available from R. T Vanderbilt Co., Inc. of Norwalk, Conn.), larch arabogalactan, gelatin, Kappa carrageenan, copolymers of maleic anhydride with ethylene or vinyl methyl ether.

A tablet according to this aspect of this invention typically does not require a lubricant to improve the flow of the powder for tablet manufacturing. However, if it is so desired a lubricant may be provided. Any lubricant known to one of ordinary skill in the art may be used. Preferred lubricants include, but are not limited to, talc, magnesium stearate, calcium stearate, stearic acid, hydrogenated vegetable oils, and carbowax in amounts of between about 2% to about 10% by weight.

Similarly, a disintegrant is not expected to be necessary to produce rapid dissolve tablets using the present tablet composition. However, a disintegrant can be included to increase the speed with which a resulting tablet dissolves in the mouth. Any disintegrant known to one of ordinary skill in the art may be used. If desired, between about 0.5% to about 1% by weight of a disintegrant can be added. Preferred disintegrants include, but are not limited to, starches, clays, celluloses, algins, gums, crosslinked polymers (including croscarmelose, crospovidone and sodium starch glycolate), VEEGUM.RTM. HV, agar, bentonite, natural sponge, cation exchange resins, aliginic acid, guar gum, citrus pulp, and sodium lauryl sulphate.

It is also generally considered unnecessary to buffer the tablet composition. However, a buffer may be beneficial in specific formulations. Any buffering agent known to one of ordinary skill in the art may be used. Preferred buffering agents include, but are not limited to, mono- and di-sodium phosphates and borates, basic magnesium carbonate and combinations of magnesium and aluminum hydroxide.

In a preferred implementation, the tableting powder is made by mixing in a dry powdered form the various components as described above, e.g., active ingredient (extract), diluent, sweetening additive, and flavoring, etc. An overage in the range of about 10% to about 15% of the active extract of the active ingredient can be added to compensate for losses during subsequent tablet processing. The mixture is then sifted through a sieve with a mesh size preferably in the range of about 80 mesh to about 100 mesh to ensure a generally uniform composition of particles.

The tablet can be of any desired size, shape, weight, or consistency. The total combined weight of an kava extract composition and a mate' extract composition in the form of a dry flowable powder in a single oral dosage is typically in the range of about 80 mg to about 600 mg. An important consideration is that the tablet is intended to dissolve in the mouth and should therefore not be of a shape that encourages the tablet to be swallowed. The larger the tablet, the less it is likely to be accidentally swallowed, but the longer it will take to dissolve or disintegrate. In a preferred form, the tablet is a disk or wafer of about ⅛ inch to about ¾ inch in diameter and about 0.2 inch to 0.08 inch in thickness, and has a weight of between about 160 mg to about 1,200 mg. In addition to disk, wafer or coin shapes, the tablet can be in the form of a cylinder, sphere, cube, or other shapes. For example, the tablet can be formed into the general shape of a mate' plant leaf. Although the tablet is preferably homogeneous, the tablet may alternatively be comprised of regions of powdered kava extract composition and mate' extract composition separated by non-kava and mate' extract regions in periodic or non-periodic sequences, which can give the tablet a speckled appearance with different colors or shades of colors associated with the kava and mate' extract regions and the non-kava and mate' extract regions

The present invention comprises methods of using compositions comprising combinations of kava extract compositions and mate' extract compositions, or kava extract compositions and theobromine compositions, disclosed herein. Methods of providing dietary supplementation are contemplated. Such compositions may further comprise vitamins, minerals and antioxidants. Compositions taught herein can also be used in methods of treatment of conditions wherein a diuretic, relaxant or vasodilator would be effective. For example, the present invention comprises methods of treatment of asthma or obstructive pulmonary disease (COPD), comprising administering an effective amount of a combination composition taught herein Methods of treatment of conditions in which a stimulant to the central nervous system would be effective or treatment of rheumatic conditions are also contemplated by the present invention.

Compositions of the present invention comprise oral delivery formulations wherein the amount of the kavalactones combined per dose is between about 0.05 mg and about 300 mg. The amount of the combined kavalactones per dose may also be between about 1 mg and about 100 mg. Compositions of the present invention may also comprise kava compounds wherein the percentages of methysticin and dihydromethysticin are reduced with an increase of kavain and dihydrokavain. In such compositions, the amount of kavain per dose is between about 0.01 mg and about 100 mg. The amount of kavain per dose may also be between about 0.5 mg and about 10 mg.

One or more of the above compositions of kava can be combined with mate' extract compositions, or with theobromine, wherein the amount of the kavalactones combined is in an amount per dose between about 0.05 mg and about 300 mg. The amount of the kavalactones in such compositions may also be between about 1 mg and about 100 mg. Compositions of kava extract and mate' extract compositions can also comprise mate' extract compositions in an amount between about 0.1 mg and about 750 mg per dose. The mate' alkaloid composition of the mate extract compositions can vary wherein caffeine is in an amount between about 0.1 and 5.0 mg, theobromine is in an amount between about 0.2 mg and 8.0 mg, and theophylline is in an amount between about 0.01 mg and 3.0 mg. Such compositions of kava extract and mate' can also be between about 10 mg and about 400 mg per dose. Finally, compositions of the present invention can comprise theobromine in an amount per dose between about 0.1 mg and 500 mg, and can also comprise theobromine in an amount per dose between about 10 mg and about 300 mg.

The combined kava and mate' or theobromine compositions may be administered daily, for one or more times, for effective treatment of acute or chronic conditions. Alternatively, separate kava and mate' compositions may be administered together for one or more times. Such compositions may be administered as a combined composition, or as separate compositions, daily for an indefinite period. One method of the present invention comprises administering at least one time a day a composition comprising kava compounds and mate' compounds. Methods also comprise administering such compositions more than one time per day, more than two times per day, more than three times per day and in a range from 1 to 15 times per day. Such administrations may be continuously, as in every day for a period of days, weeks, months or years, or may occur at specific times to treat or prevent specific conditions. For example, a person may be administered kava and mate' compositions at least once a day for years to treat obesity, or to enhance mental focus, cognition, and sense of well being.

An exemplary 250 mg tablet contains about 125.0 mg powdered kava/mate' composition, about 12.5 mg extract of Stevia, about 35.5 mg carboxymethylcellulose, and about 77.0 mg lactose. An exemplary 350 mg tablet contains about 160.0 mg powdered kava/mate' composition, about 15.0 mg extract of Stevia, about 15.0 mg acacia, and about 160.0 mg lactose. Other formulations are also possible. An exemplary tablet contains about 200 mg of kava extract composition, about 100 mg of theobromine, about 12.5 mg extract of Stevia, about 35.5 mg carboxymethylcellulose, and about 77.0 mg lactose. Another exemplary tablet contains about 100 mg of kava extract composition, about 200 mg of theobromine, about 12.5 mg extract of Stevia, about 35.5 mg carboxymethylcellulose, and about 77.0 mg lactose. Other formulations are also possible.

The present invention comprises methods of potentiating the activity of mate' or theobromine comprising administering the kava/mate' compositions disclosed herein. Methods of providing dietary supplementation are contemplated. Mate' and theobromine compositions comprise vitamins, minerals and antioxidants. Mate' and theobromine compositions can also be used in methods of treatment of conditions wherein a diuretic, relaxant or vasodilator would be effective. For example, the present invention comprises methods of treatment of asthma or obstructive pulmonary disease (COPD). Methods of treatment of conditions in which a stimulant to the central nervous system would be effective or treatment of rheumatic conditions are also contemplated by the present invention.

The compositions of the present invention are used in methods of providing antioxidant activity to cells. It is well recognized that oxygen radicals are involved in various pathologies and that antioxidants protect the cells from oxygen radical-induced damage. Pathologies that are related to oxygen radical damage include, but are not limited to, cancer, cardiovascular disorders, arthritis, inflammation and liver diseases. These and other related pathologies are treated by administering an antioxidant effective amount of a composition of the present invention in an amount less than would be necessary if treating with mate' alone.

The compositions of the present invention are also useful in treatments for obesity and as an aid for weight loss. Methods of the present invention comprise treatments for obesity and methods for enhancing weight loss comprising, administering an effective amount of a composition of the present invention, such an amount being effective in reduction of weight of an animal. The compositions of the present invention, in this and other treatment methods, may be administered daily, for one or more times, for effective treatment of acute or chronic conditions.

The present invention comprises methods for improving cognition, mental focus, and sense of well being as well as treating mental and physical fatigue and as an adjunct for weight reduction. Methods comprise administering an effective amount of the kava extract compositions and mate' extract compositions of the present invention. Methods of the present invention also comprise treatments for obesity and methods for enhancing weight loss comprising, administering an effective amount of a composition, such an amount being effective in reduction of weight of an animal. Formulations comprising oral delivery means can be administered to provide effective amounts of kava extract compounds and the mate' extract compounds or theobromine. A wide variety of oral delivery system formulations including, but not limited to, tablets, capsules, lozenges, liquids, and emulsions are contemplated by the present invention. The production of such delivery systems are readily achieved by those having skill in the art and by the methods disclosed herein.

All terms used herein are considered to be interpreted in their normally acceptable usage by those skilled in the art. Patents and patent applications or references cited herein are all incorporated by reference in their entireties.

The foregoing description includes the best presently contemplated mode of carrying out the invention. This description is made for the purpose of illustrating the general principles of the inventions and should not be taken in a limiting sense. This invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention.

### EXAMPLES

### Example 1

A kava extract composition may comprise the following:

A combination of six kavalactones in the indicated mass percentages

| | |
|---|---|
| Methysticin | 14% |
| Dihydromethysticin | 10% |
| Kavain | 42% |
| Dihydrokavain | 11% |
| Yangonin | 6% |
| Desmethoxyyangonin | 17% |

The following ingredients are mixed for the formulation:

| | |
|---|---|
| Extract of P. methysticum (from Ex.1) | 90.0 mg |
| Extract of I. paraguariensis | 150.0 mg |
| Stevioside (Extract of Stevia) | 12.5 mg |
| Carboxymethylcellulose | 35.5 mg |
| Lactose | 77.0 mg |
| Total | 365.0 mg |

The extract of I. paraguariensis comprises a ratio of theobromine to caffeine by weight of greater than 1.0. The tannin content of I paraguariensis is reduced greater than 90% by weight compared to that found in the respective native plant source.

The extract of P. methysticum comprises a ratio of kavain and dihydrokavain to methysticin and dihydromethysticin by weight of greater than 1.0. The formulations can be made into any oral dosage form and administered daily or up to 15 times per day as needed for the physiological effect (enhancement of mental focus, cognition, and sense of well-being, relaxant, weight reduction, anti-oxidant activity, diuresis, vasodilation, bronchial relaxation, asthma, COPD, and arthritis/rheumatic conditions).

### Example 2

The following ingredients were mixed for the following formulation:

| | |
|---|---|
| Extract of P. methysticum | 90.0 mg |
| Methysticum (14%) | 9.5 mg |
| Dihydromethysticum (10%) | 6.8 mg |
| Kavain (42%) | 28.4 mg |
| Dihydrokavain (11%) | 7.4 mg |
| Yangonin (6%) | 4.1 mg |
| Demethoxyyangonin (17%) | 11.5 mg |
| Extract of I. paraguariensis | 80.0 mg |
| Theobromine (51%) | 3.3 mg |
| Caffeine (44%) | 2.9 mg |
| Theophylline (5%) | 0.3 mg |
| Vitamin C | 15.0 mg |
| Sucralose | 35.0 mg |
| Mung Bean Powder 10:1 | 50.0 mg |
| Mocha Flavor | 40.0 mg |
| Chocolate Flavor | 20.0 mg |
| (RT#NV-24,397) | |
| Total | 330.0 mg |

The percentages refer to the total kavalactones and methylxanthines in the P. methysticum extract and I. paraguariensis extract, respectively. The tannin content of I. paraguariensis extract (0.6 mg) is reduced greater than 90% by weight compared to the respective natural plant source. The formulation can be made into any dosage form and administered daily up to 15 times per day as needed for the physiological effect (enhancement of mental focus, cognition, relaxation, and sense of well-being, weight reduction, anti-oxidant activity, bronchial relaxation, asthma, COPD, diuresis, vasodilation, and arthritis/rheumatoid conditions).

### Example3

The following ingredients were mixed:

| | |
|---|---|
| Extract of P. methysticum | 90.0 mg |
| Extract of I. paraguariensis | 40.0 mg |
| Theobromine | 50.0 mg |
| Caffeine | 10.0 mg |
| Theophylline | 1.0 mg |
| Vitamin C | 15.0 mg |
| Sucralose | 40.0 mg |
| Mung Bean Powder 10:1 | 30.0 mg |
| Mocha Flavor Ungerer FK4578 | 60.0 mg |
| Bitterness Blocker | 3.0 mg |
| X-base M-500 | 50.0 mg |
| X-base Xanthan Gum | 1.0 mg |
| Total | 390.0 mg |

The extract of P. methysticum comprises kavain and dihydrokavain in an amount that is greater by weight than methysticin and dihydromethysticin. The extract of I. paraguariensis comprises theobromine in an amount that is greater by weight than caffeine. The tannin content of I. paraguariensis has been reduced by greater than 90% by weight compared to the natural plant source. Although this formulation has been made as a freeze dried rapid dissolve tablet, the formulation can be made into any oral dosage form and administered up to 15 times per day as needed for the physiological effect (enhancement of cognition, mental focus, relaxation, and sense of well-being, weight reduction, anti-oxidant, bronchial dilation, asthma, COPD, diuresis, vasodilation and arthritis/rheumatoid conditions). This formulation has been used successfully to provide the beneficial effects without any deleterious secondary effects having been observed.

1. A composition, comprising a combination of a kava extract composition and a mate extract composition.

2. The composition of claim 1, wherein the kava extract composition comprises one or more kavalactones selected from kavain, dehydrokavain, methysticin, dihydromethysticin, yangonin, and demethoxyyangonin.

3. The composition of claim 1, wherein the kava extract composition comprises an amount of kavalactones between 0.1 mg and 400 mg.

4. The composition of claim 2, wherein the mass percentage of kavain and dihydrokavain is greater than 50% of the total weight of the kavalactones.

5. The composition of claim 2, wherein the mass percentage of methysticin and dihydromethysticin is less than 30% of the total weight of the kavalactones.

6. The composition of claim 2, wherein the mass percentage of yangonin and demethoxyyangonin is less than 20% of the total weight of the kavalactones.

7. The composition of claim 1, wherein the mate' extract composition comprises an amount between 0.1 mg and 750 mg.

8. The composition of claim 1, wherein the mate extract composition comprises an extract of I. paraguariensis in an amount between 0.1 mg and 500 mg, and is substantially free of caffeine.

9. The composition of claim 8, wherein the mate' extract composition is substantially free of tannins.

10. A composition comprising, a combination of a kava extract composition and theobromine, wherein the theobromine is in an amount between 0.1 mg and 500 mg.

11. A method for treating a physiological condition, comprising administering an effective amount of a composition comprising a combination of a kava extract composition and a mate extract composition.

12. The method of claim 11, wherein the kava extract composition comprises one or more kavalactones selected from kavain, dehydrokavain, methysticin, dihydromethysticin, yangonin, and demethoxyyangonin.

13. The method of claim 12, wherein the amount of combined kavalactones is between 0.1 mg and 400 mg, and the combined kavain and dihydrokavain component by mass percentage of the kavalactones is greater than 50%, the combined methysticin and dihydromethysticin component by mass percentage of the kavalactones is less than 30%, and the combined yangonin and demethoxyyangonin component by mass percentage of kavalactones is less than 20%.

14. The method of claim 11, wherein the mate' extract composition is in an amount between 0.1 mg and 750 mg.

15. The method of claim 11, wherein the mate extract composition comprises an extract of I. paraguariensis that is substantially free of caffeine, and is in an amount between 0.1 mg and 500 mg.

16. The method of claim 11, wherein the mate' extract composition comprises an extract of I. paraguariensis that is substantially free of caffeine and tannins, and is in an amount between 0.1 mg and 500 mg.

17. The method of claim 11, wherein the mate' extract composition is replaced with theobromine that is present in an amount between 0.1 mg and 500 mg.

18. The method of claim 11, wherein the condition is mental or physical fatigue, anxiety, tension, cognitive impairment, weight gain, nervous depression, psychogenic or fatigue headache, pain, diuresis, or inflammation.
